(19) United States
(12) Patent Application Publication
(10) Pub. No.: US 2004/0197779 A1
(43) Pub. Date: Oct. 7, 2004

(54) Methods for analyzing mixtures of proteins

(76) Inventor: James Alexander Apffel JR., Mountain View, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/405,527

(22) Filed: Apr. 3, 2003

(51) Int. Cl. C12Q 1/68

(52) U.S. Cl. 435/6

(57) ABSTRACT

Methods and kits are disclosed for detecting one or more proteins in a sample suspected of containing a plurality of the proteins. An assay medium comprising the sample and a capture agent for each of the proteins is incubated. Each of the capture agents comprises a protein-binding portion and a nucleic acid portion. Incubation is carried out under conditions for binding of the capture agents to the proteins to form capture agent-protein complexes. A mixture comprising the complexes is separated from the capture agents. The nucleic acid portions of the complexes in the mixture are then related to the presence or amount of one or more of the proteins in the sample.

### BACKGROUND OF THE INVENTION

This invention relates generally to methods and kits for determining proteins in a sample suspected on containing such proteins. The invention has particular application to multiplexed protein analysis.

Proteomics has come of interest over the last few years. While proteomics is more complex than genomics, the study of proteins gives more accurate pictures of cell biology than studying mRNA. The field of proteomics is very broad and involves areas such as, for example, protein profiling by the use of two-dimensional gel electrophoresis and mass spectrometry to study proteins expressed in the cell, protein-protein interaction using yeast two-hybrid method, pathway analysis to understand signal transduction and other complex cell processes, large scale protein folding and 3-D structure studies and high-throughput expression and purification of proteins, cellular expression during metabolism, mitosis, meiosis, in response to an external stimulus, e.g., drug, virus, change in physical or chemical condition, involving excess or deficient nutrients and cofactors, stress, aging, presence of particular strains of an organism and identifying the organism and strain, multiple drug resistance, protein-DNA interactions, protein-peptide interactions, and the like.

The human genome contains approximately 30,000 genes, of which 5,000-6,000 may be expressed in a given cell type. Although DNA sequencing of the human genome has been essentially completed, there remains the need to determine the functions of gene products. At the most basic level, genes code for proteins. In the past, the “central dogma” of modem biology states that a single gene codes for a single protein. However, in the last decade it has become clear that the real system is much more complex and in fact, a single gene may result in many different proteins due to alternate splicing and post-translational modifications such as glycosylation, phosphorylation, etc. Insights into gene function are provided by their expressed protein levels in different cell types, developmental stages, organism phenotypes, disease states, responses to stimuli, etc. Measuring these levels requires the initial resolution of complex mixtures of cellular proteins. Linkage of a specific gene to its protein product may then be established by sequencing or tryptic mapping of the protein and comparison with amino acid (AA) sequences predicted from DNA sequence databases.

There are a number of known methods for resolving cellular protein mixtures, one of which is two-dimensional polyacrylamide gel electrophoresis (2D PAGE), which separates polypeptides based on the orthogonal parameters of isoelectric point (pI) and size. In an alternative technique, amino acid sequence data is obtained from single peptides by tandem mass spectrometry (MS/MS) and used to screen databases for unique protein sequences. In this technique, selected peptide masses are isolated in the first stage of the spectrometer and subjected to collision-induced chemical dissociation, and the masses of the subfragments are then analyzed in the second stage to deduce the AA sequence.

Because of the time and effort required for individual protein isolation, digestion and analysis, high-throughput strategies involving direct proteolysis and peptide analysis of protein mixtures have been demonstrated (Washburn, M. P., D. Wolters, and J. R. Yates, Large-scale analysis of the yeast proteome by multidimensional protein identification technology. Nature Biotechnology, 2001. 19(3): p. 242-247). Such techniques include liquid chromatography (LC) coupled with MS/MS to separate and identify unique peptide sequences from tryptic digests of protein mixtures. Other techniques include MALDI (matrix assisted laser desorption ionization) or ESI (Electrospray Ionization) coupled with Ion Trap, Triple Quadrupole or QTOF Mass Spectrometry Analyzers. Alternatively, high resolution Fourier transform ion cyclotron resonance (FTICR)-MS may be employed to identify unique peptide masses in complex protein digests. All of the above approaches have one or more shortcomings.

Peptide and protein arrays on solid surfaces have received considerably less attention in comparison to DNA arrays. This is likely due to the inherent instability of these peptides and proteins at interfaces and in the presence of complex biological matrices as well as to the relatively complex interaction of target proteins and capture agents. For example, it is well known that many proteins denature upon contact with solid surfaces. Peptides, as well as proteins, are also subject to hydrolysis by any proteases that may be present in the biological sample being analyzed. Photolithographic techniques are commonly used (by Affymetrix, for example) for DNA arrays, but as yet not for protein arrays.

U.S. Patent Publication No. 20020055125 discloses an array of protein-binding agents stably attached to the surface of a solid support. The array includes a solid substrate having a substantially planar surface, and a plurality of different protein-binding agents bound to the substrate. Each of the protein-binding agents includes an anchoring segment stably bound to the substrate surface, a peptidomimetic protein-binding segment, and a linker segment connecting and separating the anchoring and peptidomimetic segments.

There remains a need to perform analytical and diagnostic assays for proteins on a large scale. The assays should have high sensitivity and high specificity and permit multiplexed determinations. It is desirable to have a means for identifying a large number of proteins in a single sample, as well as providing some quantitation of the different proteins being detected.

### SUMMARY OF THE INVENTION

One embodiment of the present invention is a method for detecting one or more proteins in a sample suspected of containing a plurality of the proteins. An assay medium comprising the sample and a capture agent for each of the proteins is incubated. Each of the capture agents comprises a protein-binding portion and a nucleic acid portion. Incubation is carried out under conditions for binding of the capture agents to the proteins to form capture agent-protein complexes. A mixture comprising the complexes is separated from the capture agents. The nucleic acids of the nucleic acid portions of the complexes in the mixture are then related to the presence or amount of one or more of the proteins in the sample.

Another embodiment of the present invention is a method for detecting one or more proteins in a sample suspected of containing a plurality of the proteins. An assay medium comprising the sample and a capture agent for each of the proteins is incubated under conditions for binding of the capture agents to the proteins to form capture agent-protein complexes. Each of the capture agents comprises a protein-binding portion and a nucleic acid portion. A mixture comprising the complexes is separated from the capture agents. The nucleic acids of the nucleic acid portions of the complexes are contacted with a solid surface to bind the nucleic acids to said solid surface. The solid surface is examined for the presence and/or amount of the nucleic acids of the complexes in the mixture. The presence and/or amount thereof are then related to the presence or amount of one or more of the proteins in the sample. In one embodiment the solid surface comprises an array of polynucleotides.

Another embodiment of the present invention is a kit for detecting one or more proteins in a sample suspected of containing a plurality of the proteins. The kit comprises in packaged combination a capture agent for each of the proteins and a solid surface comprising a plurality of specific binding partners, each specific for a nucleic acid portion of one of the capture agents. Each of the capture agents comprises a protein-binding portion and a nucleic acid portion. In one embodiment the solid surface comprises an array of specific binding partners. In one embodiment the kit further comprises one or more reagents for conducting an amplification of the nucleic acid portions of the capture agents. In one embodiment the kit further comprises a plurality of labeled detection agents.

### DESCRIPTION OF SPECIFIC EMBODIMENTS OF THE INVENTION

In a broad aspect the present invention uses a plurality of surface-bound nucleic acids, such as those found in nucleic acid array technology, to analyze a complex mixture of proteins in a sample. The invention further utilizes a plurality of capture agents for the proteins. Each of the capture agents comprises a protein-binding portion and a nucleic acid portion. The protein-binding portion or moiety may be, for example, a polynucleotide, an aptamer, a peptide, a polypeptide, an antibody mimic, a display protein, a peptide mimetic and the like. The nucleic acid portion is selected to have a sequence of nucleotides that binds to a particular polynucleotide that is present on the surface of a solid substrate, or vice versa. In this manner nucleic acid arrays, the technology for which is well-developed, may be employed to analyze complex protein mixtures. The difficulties associated with protein arrays for analysis of protein mixtures are avoided.

In one embodiment of the present invention, an assay medium, which comprises a sample and a capture agent for each of the proteins suspected of being in the sample, is incubated. The capture agents may be present in solid phase or solution phase, usually, solid phase. Incubation is carried out under conditions for binding of the capture agents to the proteins to form capture agent-protein complexes. A mixture comprising the complexes is separated from the capture agents. The nucleic acids of the nucleic acid portions of the complexes in the mixture are then related to the presence or amount of one or more of the proteins in the sample.

As used herein, the term “binding” refers to an interaction or complexation between a target protein and a protein-binding moiety such as an aptamer, resulting in a sufficiently stable complex so as to permit separation of complexes from uncomplexed protein-binding moieties under given binding complexation or reaction conditions. Binding is mediated through hydrogen bonding or other molecular forces.

The following discussion describes in detail the various reagents and surfaces that are employed in the practice of the present invention. The discussion is subdivided under the following headings: Capture Agents for Use in the Present Invention, Solid Substrate with Specific Binding Partners for Nucleic Acids, Methods of the Invention Using the Capture Agents, and Examples.

### Capture Agents for Use in the Present Invention

As mentioned above, the present invention utilizes particular reagents to achieve the benefits of the present methods. These reagents are capture agents for the proteins suspected of being present in a sample to be analyzed. Each of the capture agents comprises a protein-binding portion and a nucleic acid portion.

### Protein-binding Portion

The protein-binding portion of the capture agents of the present invention may be any moiety that is capable of binding to the target protein, usually binding to the target protein in a specific manner. The protein-binding moiety may be a polynucleotide such as a nucleic acid, an oligonucleotide such as, e.g., an aptamer, and so forth; a peptide; a polypeptide, e.g., a protein such as an antibody, an enzyme, etc.; an antibody mimic, such as a Pronectin™ biopolymer, etc.; a display protein such as, e.g., a ribosomal display protein, phage display protein, and so forth; a peptide mimetic; and so forth. The protein-binding moiety may be naturally occurring or may be produced by synthetic techniques such as recombinant techniques, hybridoma techniques, fusion techniques, and so forth.

Aptamer: One example of a protein-binding portion or moiety is an aptamer. As used herein, the term “aptamer” means a specific binding oligonucleotide, which is an oligonucleotide that is capable of forming a complex with an intended target protein substance. The complexation is target-specific in the sense that other materials such as other proteins that may accompany the target protein do not complex to the aptamer. It is recognized that complexation and affinity are a matter of degree; however, in this context, “target-specific” means that the aptamer binds to target with a much higher degree of affinity than it binds to contaminating materials. The meaning of specificity in this context is thus similar to the meaning of specificity as applied to antibodies, for example. The aptamer may be prepared by any known method, including synthetic, recombinant, and purification methods. Further, the term “aptamer” also includes “secondary aptamers” containing a consensus sequence derived from comparing two or more known aptamers to a given target.

In general, a minimum of approximately 9 nucleotides, preferably at least 35 nucleotides, is necessary to effect specific binding of the aptamer to a target protein. The only apparent limitations on the binding specificity of the target/aptamer complexes of the invention concern sufficient sequence to be distinctive in the binding oligonucleotide and sufficient binding capacity of the target substance to obtain the necessary interaction. Oligonucleotides of sequences shorter than 9 may also be feasible if the appropriate interaction can be obtained in the context of the environment in which the target protein is found or placed. Although the oligonucleotides of the aptamers generally are single-stranded or double-stranded, it is contemplated that aptamers may sometimes assume triple-stranded or quadruple-stranded structures.

The aptamer protein-binding moiety may be selected for each protein. An example of an approach is that described in U.S. Pat. Nos. 5,270,163 and 5,475,096. In addition, oligonucleotides that bind to a target protein may also be identified by in vitro selection. Another approach is that described by Blackwell, T. K., et al., Science (1990) 250:1104-1110; Blackwell, T. K., et al., Science (1990) 250:1149-1152; Tuerk, C., and Gold, L., Science (1990) 249:505-510; Joyce, G. F., Gene (1989) 82:83-87. Tuerk and Gold describe the use of a procedure termed “systematic evolution of ligands by exponential enrichment.” Kinzler, K. W., et al., Nucleic Acids Res. (1989) 17:3645-3653, identified double-stranded DNA sequences that were bound by proteins that bind to DNA and regulate gene expression. Thiesen, H.-J., and Bach, C., Nucleic Acids Res. (1990) 18:3203-3208, describe what they call a target detection assay (TDA) to determine double-stranded DNA binding sites for putative DNA binding proteins.

The specific binding oligonucleotides of the aptamers need to contain the sequence-conferring specificity, but may be extended with flanking regions and otherwise derivatized or modified.

The aptamers found to bind to the target proteins may be isolated, sequenced, and then re-synthesized as conventional DNA or RNA moieties, or may be “modified” oligomers, which are those conventionally, recognized in the art. These modifications include, but are not limited to incorporation of: (1) modified or analogous forms of sugars (ribose and deoxyribose); (2) alternative linking groups; or (3) analogous forms of purine and pyrimidine bases.

The aptamers may also include intermediates in their synthesis. For example, any of the hydroxyl groups ordinarily present may be replaced by phosphonate groups, phosphate groups, protected by a standard protecting group, or activated to prepare additional linkages to additional nucleotides. The 5′ terminal OH is conventionally free but may be phosphorylated; OH substituents at the 3′ terminus may also be phosphorylated. The hydroxyls may also be derivatized to standard protecting groups.

One or more phosphodiester linkages may be replaced by alternative linking groups. These alternative linking groups include, but are not limited to embodiments wherein P(O)O is replaced by P(O)S (“thioate”), P(S)S (“dithioate”), P(O)NR₂ (“amidate”), P(O)R, P(O)OR′, CO or CH₂ (“formacetal”), wherein each R or R′ is independently H or substituted or unsubstituted alkyl (1-20C.) optionally containing an ether (—O—) linkage, aryl, alkenyl, cycloalkyl, cycloalkenyl or aralkyl.

The oligonucleotides of the aptamers may contain the conventional bases adenine, guanine, cytosine, and thymine or uridine. Aptamers may be sequenced and re-synthesized as mentioned above. Included within the term aptamers are synthetic aptamers that incorporate analogous forms of purines and pyrimidines. “Analogous” forms of purines and pyrimidines are those generally known in the art, many of which are used as chemotherapeutic agents. An exemplary but not exhaustive list includes aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyl-uracil, 5-methoxyaminomethyl-2-thiouracil, beta-D-mannosylqueosine, 5-methoxyuracil, 2-methyl-thio-N6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, 5-pentynyl-uracil and 2,6-diaminopurine. The use of uracil as a substitute base for thymine in deoxyribonucleic acid (hereinafter referred to as “dU”) is considered to be an “analogous” form of pyrimidine in this invention.

Aptamer oligonucleotides may contain analogous forms of ribose or deoxyribose sugars that are generally known in the art. An exemplary, but not exhaustive list includes 2′ substituted sugars such as 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside.

One particular embodiment of aptamers that are useful in the present invention is based on RNA aptamers as disclosed in U.S. Pat. Nos. 5,270,163 and 5,475,096, the relevant portions of which are incorporated herein by reference. The aforementioned patents disclose the SELEX method, which involves selection from a mixture of candidate oligonucleotides and stepwise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with a target such as a target protein under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific, high affinity nucleic acid ligands to the target molecule.

The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions.

Another particular embodiment of aptamers that are useful in the present invention is disclosed in U.S. Pat. No. 6,001,577, the relevant disclosure of which is incorporated herein by reference. Nucleic acid ligands to target molecules are identified using the SELEX procedure wherein the candidate nucleic acids contain photoreactive groups and nucleic acid ligands identified thereby. The complexes of increased affinity nucleic acids and target molecules formed in the procedure are crosslinked by irradiation to facilitate separation from unbound nucleic acids. In other methods partitioning of high and low affinity nucleic acids is facilitated by primer extension steps in which chain termination nucleotides, digestion resistant nucleotides or nucleotides that allow retention of the cDNA product on an affinity matrix are differentially incorporated into the cDNA products of either the high or low affinity nucleic acids and the cDNA products are treated accordingly to amplification, enzymatic or chemical digestion or by contact with an affinity matrix.

Antibodies: An antibody is an immunoglobulin that specifically binds to and is thereby defined as complementary with a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

Antiserum containing antibodies (polyclonal) is obtained by well-established techniques involving immunization of an animal, such as a rabbit, guinea pig, or goat, with an appropriate immunogen and obtaining antisera from the blood of the immunized animal after an appropriate waiting period. State-of-the-art reviews are provided by Parker, Radioimmunoassay of Biologically Active Compounds, Prentice-Hall (Englewood Cliffs, N.J., U.S., 1976), Butler, J. Immunol. Meth. 7: 1-24 (1975); Broughton and Strong, Clin. Chem. 22: 726-732 (1976); and Playfair, et al., Br. Med. Bull. 30: 24-31 (1974).

Antibodies can also be obtained by somatic cell hybridization techniques, such antibodies being commonly referred to as monoclonal antibodies. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (N.Y. 1978), Nature 266: 495 (1977), Science 208: 692 (1980), and Methods of Enzymology 73 (Part B): 3-46 (1981). Samples of an appropriate immunogen preparation are injected into an animal such as a mouse and, after a sufficient time, the animal is sacrificed and spleen cells obtained. Alternatively, the spleen cells of a non-immunized animal can be sensitized to the immunogen in vitro. The spleen cells are fused with a myeloma cell line, generally in the presence of a non-ionic detergent, for example, polyethylene glycol. The resulting cells, which include fused hybridomas, are allowed to grow in a selective medium, such as HAT-medium, and the surviving immortalized cells are grown in such medium using limiting dilution conditions. The cells are grown in a suitable container, e.g., microtiter wells, and the supernatant is screened for monoclonal antibodies having the desired specificity.

Various techniques exist for enhancing yields of monoclonal antibodies, such as injection of the hybridoma cells into the peritoneal cavity of a mammalian host, which accepts the cells, and harvesting the ascites fluid. Where an insufficient amount of the monoclonal antibody collects in the ascites fluid, the antibody is harvested from the blood of the host. Alternatively, the cell producing the desired antibody can be grown in a hollow fiber cell culture device or a spinner flask device, both of which are well known in the art. Various conventional ways exist for isolation and purification of the monoclonal antibodies from other proteins and other contaminants (see Köhler and Milstein, supra).

In another approach for the preparation of antibodies the sequence coding for antibody binding sites can be excised from the chromosome DNA and inserted into a cloning vector, which can be expressed in bacteria to produce recombinant proteins having the corresponding antibody binding sites.

In general, antibodies can be purified by known techniques such as chromatography, e.g., DEAE chromatography, ABx chromatography, and the like, filtration, and so forth.

Antibody Mimics: Chemicals that mimic the functions of antibodies are known. Such antibody mimics usually are small in size, which allows them to avoid provoking an immunogenic response. There are several approaches to the structure and manufacture of antibody mimics. One approach utilizes an alternative protein framework, such as cytochrome b₅₆₂. (Hsieh-Wilson, et al., (1996), Acc. Chem Res. 29:164-170). Some of the functions of antibodies have been mimicked in structures comprising ribonucleic acids (RNA). Hiseh-Wilson, et al., (1996), supra).

Unnatural oligomers such as benzodiazepines, beta-turn mimics, protease inhibitors and purine derivatives have also been tested for their ability to function as antibody mimics. (Hsieh-Wilson, et al., (1996), supra). Unnatural biopolymers such as oligocarbamates, oligoureas and oligosulfones have been proposed as antibody mimics. (Hsieh-Wilson, et al., (1996), supra).

Molecules with some of the recognition properties of antibodies have been created by joining various substituents to scaffolds such as xanthene and cubane. These substituents include peptides but do not include peptide loops. (Hsieh-Wilson, et al., (1996), supra). Antibody mimics comprising a scaffold of a small molecule such as 3-aminomethylbenzoic acid and a substituent consisting of a single peptide loop have been constructed. The peptide loop performs the binding function in this mimic. (Smythe, et al., (1994), J. Am. Chem. Soc. 116:2725-2733)

Another example of an antibody mimic is that disclosed in U.S. Pat. No. 5,770,380. The antibody mimic has multiple peptide loops bound to an organic or molecular scaffold. In a preferred embodiment, the loops all project from the same side of the scaffold with respect to one another. The binding sites of the antibody mimic are provided by peptide loops. The potential diversity of binding sites is alleged to be far greater than with other oligomers and polymers. Because the loops project from the same side of the scaffold, all of the loops are available for binding purposes and binding ability is increased.

A particularly attractive protein-binding moiety is a Pronectin™ biopolymer, which is a synthetic antibody mimic based on fibronectins, developed by Phylos, Inc., Lexington, Mass. In the process of selecting Pronectin™ biopolymers as specific capture agents, a large library of RNA is expressed and the messages are covalently attached at the 3′ end to the product protein by puromycin. Pronectin-F™ is a recombinant protein containing multiple copies of the RGD cell attachment ligand of human fibronectin interspersed between repeated structural peptide segments derived from spider silk. Pronectin F is a trademark of Protein Polymer Technologies of San Diego, Calif.). See, for example, U.S. Pat. No. 5,514,581, the relevant disclosure of which is incorporated herein by reference.

Display Proteins: Another protein binding moiety for the capture agents of the invention is a display protein that comprises a protein and a nucleic acid such as a synthesized protein with its attendant nucleic acid portion. One example of such a display protein is ribosomal display proteins, which generally comprise one or more proteins associated with an RNA molecule. Ribosomes are ribonucleoprotein particles in which rRNA provides a majority of the mass. In the present invention capture agents may be selected from a library of expressed proteins that are still attached to their encoding message through the ribosomal complex. This may be explained more fully as follows: As the cDNA coding for a given protein is translated into amino acids and a growing linear protein sequence, the ribosome moves along the cDNA chain. If the stop codons are removed from the cDNA coding sequence, the ribosome stalls at the end of the sequence, but remains attached. Thus, the protein and the message that codes for it remain attached.

Another example of such a display protein is phage display in which the encoding message is integrated into a filamentous bacteriophage genome and the resulting expressed protein is displayed on the surface of the head of the virus. The displayed protein can be any desired sequence, but is most commonly an antibody, antibody fragment of a combinatorial peptide. Phage display is similar to PROfusion and ribosomal display in that the message remains attached to the expressed protein and, thus, allows amplification of the protein product. In phage display, the nucleic acid is not connected directly to the protein product but rather is contained within the bacteriophage particle while the protein is attached to the outside surface.

Peptide Mimetics: A peptide mimetic refers to nonpeptide synthetic polymers or oligomers that detectably interact with proteins or receptors in a manner analogous to protein-protein or protein-peptide physical and/or chemical interactions under assay conditions. Peptide mimetics are generally protease-resistant, and include, for example, oligomeric species such as peptoids, beta-peptides, and constrained cyclic molecules such as cyclic peptides and heterocyles. In some cases, the peptide mimetic may also mimic the folding of natural proteins. Peptide mimetics may comprise generally no more than about 500 mers, no more than about 100 mers, no more than about 50 mers, no more than about 40 mers, no more than about 30 mers, no more than about 20 mers, no more than about 10 mers, no more than about 7 mers, and may be about 7 to about 500 mers. See, for example, U.S. Patent Publication No. 20020055125 and references disclosed therein relating to peptide mimetic libraries and their design and synthesis, the relevant disclosures of which is incorporated herein by reference.

Nucleic acid-protein Fusion Products: The protein-binding moiety may be a protein that is generated by an in vitro or in situ transcription/translation protocol that generates protein covalently linked to the 3′ end of its own mRNA. Examples include RNA-protein fusion products, DNA-protein fusion products, synthetic nucleic acid protein fusion products, peptide nucleic acid protein fusion products, and so forth. One particular example is fusion products disclosed in U.S. Pat. No. 6,416,950, the relevant disclosure of which is incorporated herein by reference. In one disclosed approach a method for generating DNA-protein fusions involves: (a) linking a nucleic acid primer to an RNA molecule (preferably, at or near the RNA 3′ end), the primer being bound to a peptide acceptor (for example, puromycin); (b) translating the RNA to produce a protein product, the protein product being covalently bound to the primer; and (c) reverse transcribing the RNA to produce a DNA-protein fusion. A second method involves: (a) generating an RNA-protein fusion; (b) hybridizing a nucleic acid primer to the fusion (preferably, at or near the RNA 3′ end); and (c) reverse transcribing the RNA to produce a DNA-protein fusion.

### Nucleic Acid Portion

The capture agents of the present invention further comprise a nucleic acid portion, which is associated with the protein-binding portion. In general, the nucleic acid portion is selected to have a sequence of nucleotides that binds to a particular polynucleotide, such as an oligonucleotide, that is present on the surface of a solid substrate, or vice versa. In other words, there is correspondence between such sequence of the nucleic acid moiety and a polynucleotide on a solid surface. In most instances the nucleic acid portion codes for the protein-binding portion of the capture agent. As the cDNA coding for a given protein is translated into amino acids and a growing linear protein sequence, the ribosome moves along the cDNA chain. If the stop codons are removed from the cDNA coding sequence, the ribosome stalls at the end of the sequence, but remains attached. Thus, the protein and the message that codes for it remain attached. However, this is not strictly necessary as long as each capture agent has a unique “address” nucleic acid. This means that an additional address sequence could be added for a protein sequence code. If this known address sequence is left attached to the protein after translation, it can be used as an identifier for the original protein. However, this sequence, in itself, does not code for the protein and consequently could not be amplified and used to reproduce the proteins.

The nucleic acid portion of the capture agent should have little or no secondary structure under the binding conditions employed in the present methods. In this way, irrelevant interactions between the nucleic acid portion and the target proteins are avoided. It should be kept in mind that, where the protein-binding portion of the capture agent is a nucleic acid, it may have secondary structure to promote the binding between the protein-binding portion and the target protein.

The nucleic acid portion or moiety may comprise a sequence that binds to a polynucleotide and one or more sequences that do not. Preferably, the nucleic acid moiety comprises a sequence that is at least 70% complementary, at least 80% complementary, at least 90% complementary, at least 95% complementary, at least 99% complementary to a corresponding polynucleotide on a solid surface. Most preferably, the nucleic acid moiety has a sequence that is fully complementary to a polynucleotide on a solid surface. Two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. The length of such sequence is at least about 3 nucleotides, at least about 4 nucleotides, at least about 5 nucleotides, at least about 6 nucleotides, at least about 7 nucleotides, at least about 8 nucleotides, and so forth. Usually, the length of such sequence is about 6 nucleotides to about 500 nucleotides, more usually, about 20 nucleotides to about 60 nucleotides.

The nucleic acid moiety should have at least one sequence at an end thereof to permit amplification of the nucleic acid moiety. Usually, the nucleic acid moiety has a single primer pair sequence at each end to allow for amplification of the nucleic acid of the nucleic-acid binding moiety. The length of the single primer sequence is dependent on the nature of the amplification method, and the like. The single primer sequence is usually about 7 to about 60 nucleotides, more usually, about 15 to about 25 nucleotides, in length.

The manner in which the nucleic acid portion and the protein-binding portion are associated is dependent on the nature of the components such as mode of synthesis, isolation from natural sources, in vitro translation and the like. The components of the capture agents may be associated by covalent linking, macromolecular association, non-covalent linking where the linking is of sufficient strength to withstand the parameters of the present methods, and so forth. The associated components of the capture agents may also be referred to a conjugates. In certain instances, as is evident from the above discussion regarding the protein-binding moieties, the nucleic acid portion of the capture agents is already attached to the protein-binding moiety as a result of the synthesis or isolation of the protein-binding moiety. Such a situation exists, for example, for aptamers, display proteins, nucleic acid-protein fusions, and so forth. As mentioned above, aptamers are the capture agent. For Pronectin™ biopolymers, the nucleic acid portion is covalently attached to the protein-binding portion. As is evident from the disclosure herein, the nucleic acid portion and the protein-binding portion may be attached during the process of making the capture agent.

When the protein must be associated synthetically with the nucleic acid, covalent linkages are preferred. The protein can be covalently coupled to the nucleic acid in various ways as known to those skilled in the art. In one procedure, one end of the nucleic acid is modified as with a terminal transferase so as to establish a ribonucleotide end, if not already present. The ribonucleotide end can be oxidized with periodate to form terminal aldehyde groups which can undergo a Schiffs base reaction with an amino-group of the protein, followed by hydrogenation to form a stable aminomethyl linkage between the protein and nucleic acid.

Another approach involves esterifying the 2′-OH group of the nucleic acid with a saturated or unsaturated aliphatic dicarboxylic acid or anhydride. The terminal free carboxyl group is then reacted with a diaminoalkane, generally in the presence of an activator to provide the aminoalkyl derivative. The terminal primary amino group of this derivative serves as a site for further reactions with bifunctional crosslinking reagents. Generally the esterification and amidation reactions are carried out in an aqueous medium and preferably the aqueous medium for the esterification reaction is an aqueous pyridine/acetonitrile solution.

As a variant of the above method, the terminal free carboxyl group of the ester derivative is reacted with cysteamine to provide a terminal —SH group on this modified nucleic acid or with 2,2′-dithiobis(amino ethane) to provide a terminal —S—SR group, these terminal groups also serving as sites for further reactions with bifunctional crosslinking agents.

In still another approach the nucleic acid can be enzymatically modified for linking. See, for example, U.S. Pat. No. 4,587,044, the relevant portions of which are incorporated herein by reference.

Several methods have been developed for linking modified nucleic acid to a desired protein or a group of proteins. The modified nucleic acid contains a terminal aminoalkyl or thioethyl group, which is employed as a reactive site for crosslinking with the protein. Various crosslinking agents can be employed such as a homobifunctional acylating agent, a heterobifunctional crosslinking agent, and so forth.

Other methods for linking the nucleic acid portion and the protein-binding portion include ribosomal attachment, puromycin (as in the case of Pronectin™ biopolymers) and so forth.

### Solid Substrate with Specific Binding Partners for Nucleic Acids

The nucleic acid portion of the capture agents is detected using a specific binding partner for the nucleic acid. Specific binding partners include polynucleotides, which include oligonucleotides; peptide nucleic acids; antibodies for nucleic acids; synthetic capture agents; and so forth. The specific binding partners for the nucleic acids are generally bound to the surface of a solid substrate in a predetermined arrangement so that the identity of a specific binding partner at a particular location is known. In one embodiment the predetermined arrangement is an array.

Solid substrates having a plurality of surface-bound chemical entities such as antibodies, polynucleotides, e.g., oligonucleotides, and the like are well-known in the art. Preferred materials for the substrate itself are those that provide physical support for the polynucleotides that are deposited on the surface or synthesized on the surface in situ from subunits. The materials should be of such a composition that they endure the conditions of a deposition process and/or an in situ synthesis and of any subsequent treatment or handling or processing that may be encountered in the use of the particular array.

Typically, the substrate material is transparent. By “transparent” is meant that the substrate material permits signal from features on the surface of the substrate to pass therethrough without substantial attenuation and also permits any interrogating radiation to pass therethrough without substantial attenuation. By “without substantial attenuation” may include, for example, without a loss of more than 40% or more preferably without a loss of more than 30%, 20% or 10%, of signal. The interrogating radiation and signal may for example be visible, ultraviolet or infrared light. In certain embodiments, such as for example where production of binding pair arrays for use in research and related applications is desired, the materials from which the substrate may be fabricated should ideally exhibit a low level of non-specific binding during hybridization events.

The materials may be naturally occurring or synthetic or modified naturally occurring. Suitable rigid substrates may include glass, which term is used to include silica, and include, for example, glass such as glass available as Bioglass, and suitable plastics. Should a front array location be used, additional rigid, non-transparent materials may be considered, such as silicon, mirrored surfaces, laminates, ceramics, opaque plastics, such as, for example, polymers such as, e.g., poly (vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc., either used by themselves or in conjunction with other materials. The surface of the substrate is usually the outer portion of a substrate.

The material used for an array support or substrate may take any of a variety of configurations ranging from simple to complex. Usually, the material is relatively planar such as, for example, a slide. In many embodiments, the material is shaped generally as a rectangular solid. As mentioned above, multiple arrays of chemical compounds may be synthesized on a sheet, which is then diced, i.e., cut by breaking along score lines, into single array substrates. Typically, the substrate has a length in the range about 5 mm to 100 cm, usually about 10 mm to 25 cm, more usually about 10 mm to 15 cm, and a width in the range about 4 mm to 25 cm, usually about 4 mm to 10 cm and more usually about 5 mm to 5 cm. The substrate may have a thickness of less than 1 cm, or even less than 5 mm, 2 mm, 1 mm, or in some embodiments even less than 0.5 mm or 0.2 mm. The thickness of the substrate is about 0.01 mm to 5.0 mm, usually from about 0.1 mm to 2 mm and more usually from about 0.2 to 1 mm. The substrate is usually cut into individual test pieces, which may be the size of a standard size microscope slide, usually about 3 inches in length and 1 inch in width.

The surface of the substrate onto which the materials such as polynucleotides are deposited or formed may be smooth or substantially planar, or have irregularities, such as depressions or elevations. The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethylene amines, polyarylene sulfides, polysiloxanes, polyimides, polyacetates, and the like, where the polymers may be hetero- or homo-polymeric, and may or may not have separate functional moieties attached thereto (for example, conjugated). Various further modifications to the particular embodiments described above are, of course, possible. Accordingly, the present invention is not limited to the particular embodiments described in detail above.

Peptide nucleic acids (PNA) are compounds that, in certain respects, are similar to oligonucleotide analogs, however, in other very important respects their structure is very different. (see, e.g., Egholm, et al., Science, 1991, 254, 1497; Egholm, et al., J. Am. Chem. Soc., 1992, 114, 1895; Egholm, et al., J. Am. Chem. Soc., 1992, 114, 9677; (see Egholm et al., Nature, 1993, 365, 566-568 and reference cited therein and PCT applications WO 92/20702, WO 92/20703 and WO 93/12129). PNA's are synthetic molecules comprising a polyamide backbone bearing a plurality of ligands such as naturally occurring nucleobases attached to an amide backbone through a suitable linker. PNA binds both DNA and RNA to form PNA/DNA or PNA/RNA duplexes. The resulting PNA/DNA or PNA/RNA duplexes are bound with greater affinity than corresponding DNA/DNA or DNA/RNA duplexes as determined by melting temperatures (Tm). PNA's bind to both single stranded DNA and double stranded DNA. See, for example, U.S. Pat. Nos. 6,451,968, 5,539,083 and 5,612,458, the relevant disclosures of which are incorporated herein by reference.

Antibodies that selectively bind double stranded DNA or DNA-RNA hybrids have been prepared and used in the detection of duplexes formed between particular base sequences of interest. See, for example, U.S. Pat. Nos. 4,623,627 and 4,833,084, the relevant disclosures of which are incorporated herein by reference.

Polynucleotides are compounds or compositions that are polymeric nucleotides or nucleic acid polymers. The polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. The polynucleotide can have from about 2 to 5,000,000 or more nucleotides. Usually, the oligonucleotides are at least about 2 nucleotides, usually, about 5 to about 100 nucleotides, more usually, about 10 to about 50 nucleotides, and may be about 15 to about nucleotides, in length. Polynucleotides include single or multiple stranded configurations, where one or more of the strands may or may not be completely aligned with another.

A nucleotide refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “polynucleotide” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides.

The synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, and 5,869,643, EP 0294196, and elsewhere.

Polynucleotides may be deposited on the surface of a solid substrate as fully formed moieties. On the other hand, the polynucleotides may be synthesized in situ in a series of steps such as, for example, the addition of nucleotide building blocks. Such methods of fabrication are well known. See, for example, EP 0 173 356 B1, U.S. Pat. No. 5,700,637 and PCT WO 95/25116 and PCT application WO 89/10977, U.S. Pat. No. 6,242,266 (Schleifer, et al.), (U.S. Pat. No. 6,232,072) (Fisher), U.S. Pat. No. 6,180,351 (Cattell), U.S. Pat. No. 5,474,796 (Brennan), U.S. Pat. No. 6,219,674 (Fulcrand, et al.), U.S. Pat. No. 6,258,454 (Lefkowitz, et al.). The devices and methods of the present invention are particularly useful with substrates with array areas with array assemblies of polynucleotides. An array includes any one-, two- or three-dimensional arrangement of addressable regions bearing a particular polynucleotide associated with that region. An array is addressable in that it has multiple regions of different polynucleotide sequences, such that a region or feature or spot of the array at a particular predetermined location or address on the array can detect a particular target protein(s) by virtue of binding to a nucleic acid portion of the capture agents of the present invention.

An array assembly on the surface of a substrate refers to one or more arrays disposed along a surface of an individual substrate and separated by inter-array areas. Normally, the surface of the substrate opposite the surface with the arrays (opposing surface) does not carry any arrays. The arrays can be designed for testing against any type of sample, whether a trial sample, a reference sample, a combination of the foregoing, or a known mixture of proteins. The surface of the substrate may carry at least one, two, four, or at least ten, arrays. Depending upon intended use, any or all of the arrays may be the same or different from one another and each may contain multiple spots or features of polynucleotides. A typical array may contain more than ten, more than one hundred, more than one thousand or ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges.

Any of a variety of geometries of arrays on a substrate may be used. As mentioned above, an individual substrate may contain a single array or multiple arrays. Features of the array may be arranged in rectilinear rows and columns. This is particularly attractive for single arrays on a substrate. When multiple arrays are present, such arrays can be arranged, for example, in a sequence of curvilinear rows across the substrate surface (for instance, a sequence of concentric circles or semi-circles of spots), and the like. Similarly, the pattern of features may be varied from the rectilinear rows and columns of spots to include, for example, a sequence of curvilinear rows across the substrate surface (for example, a sequence of concentric circles or semi-circles of spots), and the like. The configuration of the arrays and their features may be selected according to manufacturing, handling, and use considerations.

Each feature, or element, within the molecular array is defined to be a small, regularly shaped region of the surface of the substrate. The features are arranged in a predetermined manner. Each feature of an array usually carries a predetermined polynucleotide or mixtures thereof. Each feature within the molecular array may contain a different molecular species, and the molecular species within a given feature may differ from the molecular species within the remaining features of the molecular array. Some or all of the features may be of different compositions. Each array may contain multiple spots or features and each array may be separated by spaces or areas. It will also be appreciated that there need not be any space separating arrays from one another. Interarray areas and interfeature areas are usually present but are not essential. As with the border areas discussed above, these interarray and interfeature areas do not carry any polynucleotide. Interarray areas and interfeature areas typically will be present where arrays are formed by the conventional in situ process or by deposition of previously obtained moieties, as mentioned above. It will be appreciated though, that the interarray areas and interfeature areas, when present, could be of various sizes and configurations.

### Methods of the Invention Using the Capture Agents

The following general discussion of methods is by way of illustration and not limitation. One skilled in the art will be able to apply the technology herein in other methods for conducting protein assays. In general and as mentioned above, in the present methods a sample to be analyzed is combined with a set of capture agents of the invention. The sample may be a body fluid or a non-body fluid. The phrase “body fluid” refers to any fluid obtained from the body of a mammal (e.g., human, monkey, mouse, rat, rabbit, dog, cat, sheep, cow, pig, and the like) that is suspected of containing a particular target protein or proteins to be detected. Body fluids include, for example, whole-blood, plasma, serum, interstitial fluid, sweat, saliva, urine, semen, blister fluid, inflammatory exudates, stool, sputum, cerebral spinal fluid, tears, mucus, and the like, collection fluids used to collect proteins from protein-containing materials such as biological tissue and the like, and so forth. The biological tissue includes excised tissue from an organ or other body part of a host.

The phrase “non-body fluid” refers to any fluid not obtained from the body of a mammal, which is suspected of containing a particular target protein or proteins to be detected. Exemplary non-body fluids include cell-culture media, dialysate, and the like. The protein sample can be examined directly or may be pretreated to render the protein analyte more readily accessible to the protein-binding moiety of the capture agents of the invention.

For determining a mixture of proteins, one may use intact cells, intact viruses, viral infected cells, lysates, plasmids, mitochondria or other organelles, fractionated samples, or other aggregation of proteins, separated proteins, and treated proteins, by themselves or in conjunction with other compounds. Any source of a mixture of proteins can be used, where there is an interest in identifying a plurality of proteins. Protein analytes may be isolated using precipitation, extraction, and chromatography. The proteins may be present as individual proteins or combined in various aggregations, such as organelles, cells, viruses, etc. Protein analytes may be released from cells, for example, by lysing the cells.

As mentioned above, in the method, the sample is combined in a suitable assay medium with a set of capture agents of the invention where the protein-binding portion of each capture agent is selected to bind a particular protein of interest and the nucleic acid portion is selected to bind to a particular polynucleotide on a solid surface. The medium is usually an aqueous buffered medium at a moderate pH, generally that which provides optimum sensitivity in the present method. The aqueous medium may be solely water or may include from 0.01 to 80 or more volume percent of a cosolvent. The pH for the medium will usually be in the range of about 4 to 13, more usually in the range of about 5 to 10, and preferably in the range of about 6.5 to 9.5. The pH is generally selected to achieve optimum assay sensitivity and specificity. Among the factors that must be considered are the pH dependence of the rates of the reactions involved, the binding of binding members and the minimization of non-specific binding, and so forth.

Various buffers may be used to achieve the desired pH and maintain the pH during the determination. Illustrative buffers include borate, phosphate, carbonate, tris, barbital and the like. The particular buffer employed is not critical to this invention, but in an individual assay one or another buffer may be preferred. Various ancillary materials may be employed in the method in accordance with the present invention. For example, in addition to buffers the medium may comprise stabilizers for the medium and for the reagents employed. Frequently, in addition to these additives, proteins may be included, such as albumins; organic solvents such as formamide; quaternary ammonium salts; polyanions such as dextran sulfate; surfactants, particularly non-ionic surfactants; binding enhancers, e.g., polyalkylene glycols; or the like.

One or more incubation periods may be applied to the medium. The medium is usually incubated at a temperature and for a time sufficient for binding of the protein-binding moieties to the proteins that may be present in the sample. The incubation period is generally carried out under conditions sufficient to ensure an efficient and quantitatively reproducible binding equilibrium between the aforementioned species. Moderate temperatures are normally employed for carrying out the method and usually constant temperature, preferably, room temperature, during the period of the measurement. Incubation temperatures normally range from about 5° to about 99° C., usually from about 15° C. to about 70° C., more usually 20° C. to about 45° C. The time period for the incubation is dependent on, for example, the kinetics of the reactions between the capture agents and the target proteins, and so forth and is about 0.2 seconds to about 12 hours, usually, from about 2 seconds to 1 hour, more usually, about 10 to about 30 minutes. The time period depends on, among others, the temperature of the medium and the rate of binding of the proteins and the protein-binding moieties, which is determined by the association rate constant, the concentration, the binding constant and dissociation rate constant.

The concentration of proteins to be detected will generally vary from about 10⁻¹⁵ M to about 10⁻¹² M, more usually from about 10⁻¹⁵ M to about 10⁻¹⁴ M. In some circumstances, a predetermined cut-off level may be established for one or more of the protein analytes suspected of being in a sample. The particular predetermined cut-off level generally is determined on a case-by-case basis by one skilled in the art.

The concentration of the capture agents is dependent on the suspected concentrations of the proteins of interest in the sample, the stability of the capture agent, the solubility of the reagents, the characteristics of the binding surfaces, and so forth. The final concentration of any of the reagents will normally be determined empirically to optimize the sensitivity of the method. The concentration of the capture agents is generally about 10⁻¹² M to about 10⁻⁶M, usually, about 10⁻⁹ M to about 10⁻¹⁶M.

Following the incubation period(s), capture agent-target complexes may be separated from the medium containing the complexes and the unbound capture agents. It should be noted that it is not necessary to separate the complexes from unbound target proteins since the latter are not detected in subsequent steps. Any convenient separation means may be employed to separate the complexes from the unbound capture agents. The separation may be based on size, solubility, hydrophobicity, and the like. In principle, any technique that would allow a class separation of the complex of capture agent and target from unbound capture agent would suffice. Accordingly, in this embodiment the capture agents should all be of similar size. By the term “similar size” is meant that the hydrodynamic volume of the capture agents are uniformly small enough compared capture agent/target complex that the two can be completely separated by filtration, size exclusion chromatography or a similar technique. A number of approaches based on size may be employed for separating the complexes from the unbound capture agents. Such approaches include filtration, size exclusion chromatography, centrifugation, field flow fractionation and so forth. For example, an ultrafiltration spin column can be used in which the nominal molecular weight cutoff is smaller that the molecular weight of the complexes and larger than the capture agents. Thus, the complexes are retained while the unbound capture agents washed through. Other approaches include, for example, size exclusion chromatography, which discriminates between molecules based on their hydrodynamic radius.

The separation of the capture agent-protein complexes from the unbound capture agents may also include washing the separated complexes. The conditions for washing the complexes should not result in denaturing the complexes. The wash medium may be an aqueous medium as discussed above. For non-denaturing conditions the pH of the medium is usually about 6.5 to about 7.5, the temperature of the wash medium is usually about 25 to about 60° C., usually about 37° C., and time period is usually about 10 minutes to about 1 hour. To avoid denaturing, the aqueous wash medium should not contain urea, guanidine, detergents, or organic solvents, and so forth in an amount sufficient to denature the complexes. The resulting mixture contains only the capture agents that correspond to target proteins that were present in the original sample. The concentration of each capture agent is proportional to the concentration of the protein in the original sample. Each nucleic acid portion of the capture agents corresponds to a particular protein in the sample by virtue of the protein-binding portion of that capture agent. Each polynucleotide on a solid surface used to analyze the above mixture in turn corresponds to a particular nucleic acid of the capture agents. In this manner the identity and concentration of a particular protein in a sample may be determined.

It is often desirable to increase the concentration of, or amplify, the nucleic acids of the nucleic acid portion of the capture agent. The concentration may be enhanced by employing an amplification technique as known in the art. Any method that results in the formation of one or more copies of a nucleic acid or polynucleotide molecule or in the formation of one or more copies of the complement of a nucleic acid or polynucleotide molecule may be employed. The amplification techniques may be exponential or linear. Exponential amplification is any method that depends on the product-catalyzed formation of multiple copies of a nucleic acid or polynucleotide molecule or its complement. Linear amplification is any method that depends on the self-catalyzed formation of one or more copies of the complement of only one strand of a nucleic acid or polynucleotide molecule. Thus, the primary difference between linear amplification and exponential amplification is that the latter is autocatalyzed, that is, the product serves to catalyze the formation of more product, whereas in the former process the starting sequence catalyzes the formation of product but is not itself replicated. Examples of suitable amplification techniques, by way of illustration and not limitation, include Polymerase Chain Reaction (PCR) (U.S. Pat. Nos. 4,683,195 and 4,683,202), asymmetric PCR, the Ligase Chain Reaction (LCR) or oligonucleotide ligation assay (OLA) (U.S. Pat. Nos. 5,185,243, 5,679,524 and 5,573,907), transcriptional amplification by an RNA polymerase, rolling circle amplification, strand displacement amplification (SDA) (U.S. Pat. Nos. 5,455,166 and 5,130,238), Nucleic Acid Sequence Based Amplification (NASBA) (U.S. Pat. No. 5,409,818), cycling probe technology (CPT) (U.S. Pat. Nos. 5,011,769, 5,403,711, 5,660,988, and 4,876,187), invasive cleavage techniques such as Invader™ technology (U.S. Pat. Nos. 5,846,717; 5,614,402; 5,719,028; 5,541,311; and 5,843,669), Q-Beta replicase technology, and so forth. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, allelic PCR (see Newton et al. Nucl. Acid Res. 17:2503 91989); “reverse transcriptase PCR” or “RT-PCR”, “biotin capture PCR”, “vectorette PCR”. “panhandle PCR”, and “PCR select cDNA subtraction”, among others. The details of the aforementioned amplification procedures are well known in the art and will not be repeated here.

Preferably, the amplification method is carried out in such a manner as to incorporate a label into the nucleic acid amplification products. One simple expedient is to attach a label to one or more of the nucleotide triphosphates or oligonucleotide primers used in the amplification technique. The label is capable of being detected directly or indirectly. In general, any reporter molecule that is detectable can be a label. Labels include, for example, (i) reporter molecules that can be detected directly by virtue of generating a signal, (ii) specific binding pair members that may be detected indirectly by subsequent binding to a cognate that contains a reporter molecule, (iii) mass tags detectable by mass spectrometry, (iv) oligonucleotide primers that can provide a template for amplification or ligation and (v) a specific polynucleotide sequence or recognition sequence that can act as a ligand such as for a repressor protein, wherein in the latter two instances the oligonucleotide primer or repressor protein will have, or be capable of having, a reporter molecule and so forth. The reporter molecule can be a catalyst, such as an enzyme, a polynucleotide coding for a catalyst, promoter, dye, fluorescent molecule, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, a mass tag that alters the weight of the molecule to which it is conjugated for mass spectrometry purposes, and the like. Preferably, the label is selected from electromagnetic or electrochemical materials. In a particularly preferred embodiment, the detectable label is a fluorescent dye. Other labels and labeling schemes will be evident to one skilled in the art base on the disclosure herein.

In one approach PCR amplification can be used to generate PCR amplification products incorporating a detectable label. Thus, PCR amplification is preferably accomplished in the context of the present invention by utilizing at least one primer containing a detectable label such as a fluorescent label. In another approach a fluorescent label may be incorporated in a transcription-based system to produce labeled nucleic acid products.

Following amplification of the nucleic acid moieties of the capture agents, the resulting amplified mixture is examined to determine the presence and/or amount of the nucleic acids present in the amplified mixture, which is related to the presence and/or amount of the target proteins in the original sample. In one approach the amplified mixture is contacted with a substrate comprising a plurality of polynucleotides as described above. The solid substrate and the nucleic acids mixture are maintained in contact for a period of time and under conditions sufficient for the desired hybridization reactions to the polynucleotides on the solid substrate to occur. The conditions for hybridization reactions, such as, for example, period of time of contact, temperature, pH, salt concentration and so forth, are well known in the art and will not be repeated here.

In some embodiments the nucleic acid amplification products will not comprise a detectable label. In this instance labeled oligonucleotide probes may be employed for binding to the nucleic acid amplification products bound to the solid surface. The labels employed may be those described above. Alternatively, labeled antibodies to nucleic acid duplexes may be used or labeled antibodies to PNA/nucleic acid duplexes may be employed where PNA's are present on the solid surface.

After the appropriate period of time of contact between the solid surface and the nucleic acid amplification products, and additionally with labeled oligonucleotides if appropriate, the contact is discontinued. The solid surface may be washed to remove unbound materials. The substrate is moved to an examining device where the surface of the substrate on which the plurality of polynucleotides are present, preferably in the form of an array, is interrogated. The examining device may be a scanning device involving an optical system.

Reading of the array may be accomplished by illuminating the array and reading the location and intensity of resulting fluorescence at each feature of the array. For example, a scanner may be used for this purpose where the scanner may be similar to, for example, the AGILENT MICROARRAY SCANNER available from Agilent Technologies Inc, Palo Alto, Calif. Other suitable apparatus and methods are described in U.S. patent applications: Ser. No. 09/846,125 “Reading Multi-Featured Arrays” by Dorsel, et al.; and U.S. Pat. No. 6,406,849, the relevant portions of which are incorporated herein by reference. However, arrays may be read by methods or apparatus other than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,251,685 and 6,221,583 and elsewhere). Results from the reading may be raw results (such as fluorescence intensity readings for each feature in one or more color channels) or may be processed results such as obtained by rejecting a reading for a feature that is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

In another particular embodiment, the method is carried out under computer control, that is, with the aid of a computer. For example, an IBM® compatible personal computer (PC) may be utilized. The computer is driven by software specific to the methods described herein. The preferred computer hardware capable of assisting in the operation of the methods in accordance with the present invention involves a system with at least the following specifications: Pentium® processor or better with a clock speed of at least 100 MHz, at least 32 megabytes of random access memory (RAM) and at least 80 megabytes of virtual memory, running under either the Windows 95 or Windows NT 4.0 operating system (or successor thereof).

Software that may be used to carry out the methods may be, for example, Microsoft Excel or Microsoft Access, suitably extended via user-written functions and templates, and linked when necessary to stand-alone programs that perform homology searches or sequence manipulations. Examples of software or computer programs used in assisting in conducting the present methods may be written, preferably, in Visual BASIC, FORTRAN and C⁺⁺. It should be understood that the above computer information and the software used herein are by way of example and not limitation. The present methods may be adapted to other computers and software. Other languages that may be used include, for example, PASCAL, PERL or assembly language.

A computer program may be utilized to carry out one or more steps of the present invention. Another aspect of the present invention is a computer program product comprising a computer readable storage medium having a computer program stored thereon which, when loaded into a computer, performs the aforementioned method.

One aspect of the invention is the product of the above method, namely, the assay result, which may be evaluated at the site of the testing or it may be shipped to another site for evaluation and communication to an interested party at a remote location if desired. By the term “remote location” is meant a location that is physically different than that at which the results are obtained. Accordingly, the results may be sent to a different room, a different building, a different part of city, a different city, and so forth. Usually, the remote location is at least about one mile, usually, at least ten miles, more usually about a hundred miles, or more from the location at which the results are obtained. The data may be transmitted by standard means such as, e.g., facsimile, mail, overnight delivery, e-mail, voice mail, and the like.

“Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

Another aspect of the present invention relates to kits useful for conveniently performing the methods of the invention to analyze complex mixtures of proteins. To enhance the versatility of the subject invention, the reagents can be provided in packaged combination, in the same or separate containers, so that the ratio of the reagents provides for substantial optimization of the method and assay. The reagents may each be in separate containers or various reagents can be combined in one or more containers depending on the cross-reactivity and stability of the reagents.

A kit comprises in packaged combination a capture agent for each of the proteins and a solid surface comprising a plurality of specific binding partners, each specific for a nucleic acid portion of one of the capture agents. Each of the capture agents comprises a protein-binding portion and a nucleic acid portion. In one embodiment the solid surface comprises an array of specific binding partners. In one embodiment the kit further comprises one or more reagents for conducting an amplification of the nucleic acids of the capture agents. In one embodiment the kit further comprises a plurality of labeled detection agents.

The relative amounts of the various reagents in the kits can be varied widely to provide for concentrations of the reagents that substantially optimize the reactions that need to occur during the present method and to further substantially optimize the sensitivity of the assay. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. The kit can further include a written description of a method in accordance with the present invention as described above.

One of the advantages of the present invention is that existing polynucleotide array technology can be utilized to analyze complex mixtures of proteins. Accordingly, the present invention avoids using solid substrates with an array of proteins on the surface. In the present invention solution phase binding between capture agents and target proteins may be used, which is more rapid and efficient than solid phase binding. Direct labeling of proteins is avoided. The incorporation of a label into nucleic acid amplification products leads to signal amplification. Non-specific binding of proteins to surfaces is avoided. There is no need for target proteins to contact the surface of the solid substrate used in the present methods.

It should be understood that the above description is intended to illustrate and not limit the scope of the invention. Other aspects, advantages and modifications within the scope of the invention will be apparent to those skilled in the art to which the invention pertains. The following examples are put forth so as to provide those of ordinary skill in the art with examples of how to make and use the method and products of the invention and are not intended to limit the scope of what the inventors regard as their invention.

### EXAMPLE

One example of a method in accordance with the present invention involves the analysis of cytokines in a complex biological sample such as human serum. A group of independent antibody mimics as described in Xu, L., et al., Directed evolution of high-affinity antibody mimics using mRNA display. Chem Biol, 2002. 9(8):933 are selected from a library such that each has a high affinity for a given target protein and low affinity for all others. The analysis is directed to a set of 10 target proteins (Target Proteins) including Tumor Necrosis Factor-Alpha (TNF-Alpha), Tumor Growth Factor-Beta 1 (TGF-B1), Interferon-Gamma (IFN-G), Interleukin 1alpha (IL-1A), Interleukin 1Beta (IL-1B), Interleukin 2 (IL-2)), Interleukin 4 (IL-4), Interleukin 5 (IL-5), Interleukin 6 (IL-6), Basic Fibroblast Growth Factor (FGF-B) and Vascular Endothelial Growth Factor (VEGF). For each of these Target Proteins, there is obtained mRNA linked to an antibody mimic (as mentioned above) (Capture Agent), a set of oligonucleotide probes (Reporter Probes) of approximately 25 nucleotides in length with sequences complimentary to the mRNA expression portion of the Capture Agent and a Oligonucleotide Array constructed of oligonucleotides with sequences identical to the mRNA portions of the Capture Agent and complementary to the Reporter Probes. The Capture Agent is prepared by linking the mRNA to the antibody mimic by a puromycin covalent linkage as described in Xu, et al, supra. The set of oligonucleotide probes (Reporter Probes) of approximately 25 nucleotides in length with sequences complimentary to the mRNA expression portion of the Capture Agent is prepared by conventional nucleic acid synthesis.

The Oligonucleotide Array constructed of oligonucleotides with sequences identical to the mRNA portions of the Capture Agent and complementary to the Reporter Probes is prepared by conventional approaches to DNA array technology. These include manual spotting, robotic pin-spotting, inkjet deposition, in situ synthesis or photolithographic methods. These arrays are conventionally produced on glass substrates coated with ionic or covalent binding moieties such as Poly Lysine.

Step 1. Sample Preparation.

To prepare the sample for analysis, the complex biological matrix is purified such that the Target Proteins are not perturbed (e.g., in their native conformation), but any interfering background components such as salts, high level background proteins (such as serum albumin), proteolytic enzymes, nucleases and nucleic acids are removed or inactivated. Different types of samples will require different treatment. In this particular example, precipitation is employed:

1. Adjust the concentration and pH of the crude protein solution to the optimal values.

2. Add ammonium sulfate at an appropriate concentration. Incubate until precipitate forms.

3. Collect the precipitate and supernatant.

Alternatively or in addition to the above “salting out” procedure, the proteins of interest may be purified from smaller components (such as salts) and larger components (such as DNA) by a two step equilibrium dialysis with the first step using a low (e.g., 3500 MW) molecular weight cutoff membrane and the second step a high (e.g., 100 kDA MW) molecular weight cutoff dialysis membrane. The samples can be dialyzed in both cases against optimal buffers for binding such as, for example, phosphate buffered saline (PBS) containing protease inhibitors

Step 2: Capture Agent Preparation

Before performing the in-solution complex formation between Capture Agent and target, the Capture Agents are loaded with Reporter Probes. This consists of mixing all of the Capture Agents together at a concentration of approximately 10 pmole/μl and mixing the resulting cocktail with a mixture of Reporter Probes in 10 times excess for each Capture agent. At this point, the Reporter Probes are allowed to hybridize with the nucleic acid portion of the Capture Agent.

Step 3: Target Capture.

The reagent cocktail prepared in Step 2 above is then mixed with an equal volume of sample as prepared in Step 1 and incubated for a period of time and under conditions to allow specific complexes to form. This equilibrium is generally optimized and usually requires 1 to 3 hours. During this procedure, buffer and temperature conditions are fixed to maintain integrity of the proteins and complexes while allowing the optimal establishment of a robust equilibrium.

Step 4. Target Protein/Capture Agent Complex Isolation.

Once the Target Proteins are captured, the resulting Target Protein/Capture Agent complex is isolated from any quantitatively residual Capture Agent and Reporter Probes. (Note that in the process, many extraneous non-target proteins are also removed, but this doesn't matter since they are not associated with Reporter Probes through Capture Target complexes.) There are a number of ways to accomplish this, but for simplicity, the use of size exclusion chromatography is employed. For the present example, the Capture Agent is approximately identical in molecular weight at about 75 kDA. The Target Proteins vary in molecular weight from 15 to 30 kDa. The Reporter Probes are nearly identical in molecular weight at about 7 kDA. Thus, using a chromatographic mobile phase composition and temperature identical to the binding conditions (e.g., PBS at 40° C.), the mixture is subjected to chromatography. The earliest eluting components are collected consisting of complexes of Target Protein/Capture Agent/Reporter Probe. All later eluting fractions are discarded, consisting of residual Capture Agent/Reporter Probe Complexes and residual Reporter probes.

The collected fraction is then heated to 95° C. for 10 minutes to release the Reporter Probe from the Capture Agent. It may be necessary to physically separate the components by size exclusion chromatography or ultrafiltration, or it may be possible to use the probes directly (in the presence of the released Capture Agent). At this point, each Target Protein molecule in the original sample is represented by a single copy of a Reporter Probe.

Step 5: Quantitation of Target Abundance.

The actual quantitation of the Target Proteins is done through the Reporter Probes as proxies using DNA Oligo Array Technology. This can be done in two ways. If the Target Proteins are sufficiently concentrated in the original sample, the Reporter Probes can be hybridized directly to an appropriate oligonucleotide array and quantitated by the fluorescence emission of an appropriate fluorophore. Alternatively, if more sensitivity is required, the reporter gene can be amplified using a linear nucleic acid amplification technique. The resulting products are then analyzed and quantitated using DNA array technology. As another alternative, the Reporter Probe may be analyzed by a method such as the TaqMan® method. These techniques are well known in the field and will not be expanded on here.

All publications and patent applications cited in this specification are herein incorporated by reference as if each individual publication or patent application were specifically and individually indicated to be incorporated by reference, except insofar as they may conflict with those of the present application (in which case the present application prevails). Methods recited herein may be carried out in any order of the recited events, which is logically possible, as well as the recited order of events.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for detecting one or more proteins in a sample suspected of containing a plurality of said proteins, said method comprising:
(a) incubating an assay medium comprising said sample and a capture agent for each of said proteins, wherein each of said capture agents comprises a protein-binding portion and a nucleic acid portion, under conditions for binding of said capture agents to said proteins to form capture agent-protein complexes,
(b) separating a mixture comprising said complexes from said capture agents,
(c) relating the nucleic acid portions of said complexes in said mixture to the presence or amount of one or more of said proteins in said sample.

2. A method according to claim 1 wherein each of said capture agents is independently selected from the group consisting of aptamers, Pronectin™ biopolymer-nucleic acid conjugates, antibody-nucleic acid conjugates, peptide-nucleic acid conjugates, antibody mimic-nucleic acid conjugates, display protein-nucleic acid conjugates, and peptide mimetic-nucleic acid conjugates.

3. A method according to claim 1 wherein said nucleic acid portions of said complexes are detected with specific binding partners for said nucleic acid portions.

4. A method according to claim 3 wherein said specific binding partners for said nucleic acid portions are selected from the group consisting of polynucleotides, antibodies, and peptide nucleic acids.

5. A method accordingly to claim 1 wherein in step (c) said complexes are contacted with a solid surface on which said nucleic acid portions of said complexes are specifically detected.

6. A method according to claim 5 wherein said solid surface comprises a plurality of specific binding partners for said nucleic acid portions.

7. A method according to claim 6 wherein said specific binding partners are polynucleotides.

8. A method according to claim 6 wherein said specific binding partners are in a predetermined arrangement on said solid surface.

9. A method according to claim 6 wherein said solid surface comprises an array of said specific binding partners.

10. A method according to claim 1 wherein said complexes are separated from said capture agents by size.

11. A method for detecting one or more proteins in a sample suspected of containing a plurality of said proteins, said method comprising:
(a) incubating an assay medium comprising said sample and a capture agent for each of said proteins, wherein each of said capture agents comprises a protein-binding portion and a nucleic acid portion, under conditions for binding of said capture agents to said proteins to form capture agent-protein complexes,
(b) separating a mixture comprising said complexes from said capture agents,
(c) contacting the nucleic acid portions of said complexes with a solid surface such that said nucleic acids become bound thereto, and
(d) examining said solid surface for the presence and/or amount of said nucleic acid portions of said complexes in said mixture and relating the presence or amount thereof to the presence or amount of one or more of said proteins in said sample.

12. A method according to claim 11 wherein each of said capture agents is independently selected from the group consisting of aptamers, Pronectin™ biopolymer-nucleic acid conjugates, antibody-nucleic acid conjugates, peptide-nucleic acid conjugates, antibody mimic-nucleic acid conjugates, display protein-nucleic acid conjugates, and peptide mimetic-nucleic acid conjugates.

13. A method according to claim 11 wherein said surface comprises a plurality of polynucleotides arranged as features on said solid surface.

14. A method according to claim 11 wherein said polynucleotides are oligonucleotides.

15. A method according to claim 11 wherein said polynucleotides are DNA or RNA.

16. A method according to claim 11 wherein said complexes are separated from said capture agents by size.

17. A method according to claim 11 wherein said nucleic acid portions of said complexes are amplified prior to step (c).

18. A method according to claim 17 wherein said nucleic acid portions are amplified by a linear amplification method.

19. A method according to claim 17 wherein a label is incorporated into said amplified nucleic acid portions.

20. A method according to claim 19 wherein said label is fluorescent.

21. A method according to claim 11 wherein said solid surface is examined with a labeled detection agent.

22. A kit for detecting one or more proteins in a sample suspected of containing a plurality of said proteins, said kit comprising in packaged combination:
(a) a capture agent for each of said proteins, wherein each of said capture agents comprises a protein-binding portion and a nucleic acid portion, and
(b) a solid surface comprising a plurality of specific binding partners, each specific for a nucleic acid portion of one of said capture agents.

23. A kit according to claim 22 wherein each of said capture agents is independently selected from the group consisting of aptamers, Pronectin™ biopolymer-nucleic acid conjugates, antibody-nucleic acid conjugates, peptide-nucleic acid conjugates, and antibody mimic-nucleic acid conjugates.

24. A kit according to claim 22 wherein said specific binding partners for said nucleic acid portions are selected from the group consisting of polynucleotides, antibodies, and peptide nucleic acids.

25. A kit accordingly to claim 22 wherein said specific binding partners are in a predetermined arrangement on said solid surface.

26. A kit according to claim 22 wherein said solid surface comprises an array of said specific binding partners.

27. A kit according to claim 22 wherein said specific binding partners are DNA or RNA.

28. A kit according to claim 22 further comprising one or more reagents for conducting an amplification of said nucleic acid portions of said capture agents.

29. A kit according to claim 28 wherein said amplification is linear amplification.

30. A kit according to claim 28 wherein one of said reagents comprises a label for incorporation into amplified nucleic acids.

31. A kit according to claim 30 wherein said label is fluorescent.

32. A kit according to claim 22 further comprising a plurality of labeled detection agents.
